(19) United States
(12) Patent Application Publication
(10) Pub. No.: US 2022/0154145 A1
(43) Pub. Date: May 19, 2022

(54) SICKLE CELL POTENCY ASSAY

(71) Applicant: bluebird bio, Inc., Cambridge, MA (US)

(72) Inventors: ILYA SHESTOPALOV, BILLERICA, MA (US); MELISSA BONNER, NATICK, MA (US); GRETCHEN AMBERLEA MAI LEWIS, CAMBRIDGE, MA (US)

(73) Assignee: bluebird bio, Inc., Cambridge, MA (US)

(21) Appl. No.: 17/440,543

(22) PCT Filed: Mar. 20, 2020
(86) PCT No.: PCT/US2020/024025

§ 371 Date: Sep. 17, 2021

Related U.S. Application Data
(60) Provisional application No. 62/821,983, filed on Mar. 21, 2019.

(51) Int. Cl. C12N 5/078; C12N 15/86; G01N 1/30; G01N 15/14

(52) U.S. Cl. CPC C12N 5/0641; C12N 15/86; G01N 1/30; G01N 15/1434; G01N 2015/1006; C12N 2506/03; G01N 2001/305; C12N 2500/02; G01N 2001/302; C12N 2740/15043

(57) ABSTRACT

Disclosed herein are potency assays for a gene therapy treatment for sickle cell disease. Also disclosed herein are methods for measuring relative potency of a drug product used for the treatment of sickle cell disease.

### RELATED APPLICATIONS

This application claims the benefit of U.S. Provisional Application No. 62/821,983, filed Mar. 21, 2019. The entire teachings of the above application are incorporated herein by reference.

### BACKGROUND OF THE INVENTION

Sickle Cell Disease (SCD) is caused by a single point mutation in the human beta-globin gene that leads to the production of sickled hemoglobin (HbS) in erythroid cells. Under low oxygen conditions, HbS polymerizes and causes red blood cells (RBCs) to morphologically change to the characteristic “sickled” shape that is responsible for much of the pathophysiology in SCD. Early clinical results with ex vivo cell-based gene therapy have shown promise in SCD (Ribeil, et al. The New England Journal of Medicine, 2017), and as these and other cell-based therapies advance there is a need to objectively quantify drug product potency.

### SUMMARY OF THE INVENTION

Disclosed herein are potency assays for a gene therapy treatment for sickle cell disease (SCD). The potency assays comprise transducing a population of hematopoietic stem or progenitor cells from a subject that has sickle cell disease with a lentiviral vector comprising a polynucleotide encoding a globin; performing two-phase erythroid differentiation of the population of hematopoietic stem or progenitor cells comprising culturing the hematopoietic stem or progenitor cells under hypoxia during erythroid differentiation; fixing and staining the differentiated erythroid cells; analyzing the fixed and stained erythroid cells with an imaging device; calculating a Sickle Index value for the analyzed erythroid cells; and calculating the percent of sickled erythroid cells in the population, wherein the potency of the gene therapy treatment is the proportion of sickled cells in the cell population relative to an untransduced control.

In some embodiments, the potency assay further comprises obtaining the hematopoietic stem or progenitor cells from the subject that has sickle cell disease. In some embodiments, the hematopoietic stem or progenitor cells comprise CD34⁺ cells, CD133+ cells, or CD34+CD38^{Lo}CD90+CD45RA⁻ cells. In some embodiments, the hematopoietic stem or progenitor cells comprise a pair of β-globin alleles selected from the group consisting of: β^{E}/β^{S}, β⁰/β^{S}, β^{C}/β^{S}, β⁺/β^{S} and β^{S}/β^{S}. In some embodiments, the globin is a human β-globin, a human β-globin, an anti-sickling globin, a human γ-globin, a human β^{A-T87Q}-globin, a human β^{A-G16D/E22A/T87Q}-globin, or a human β^{A-T87Q/K95E/K120E}-globin protein. In some embodiments, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, or a derivative thereof.

In some embodiments, erythroid differentiation occurs in HiF erythroid differentiation media. Erythroid differentiation may occur for a period of 21 to 25 days, or for a period of 21 days. In some embodiments, the culturing of cells in the first phase of erythroid differentiation occurs under normoxia conditions. The culturing of cells in the first phase of erythroid differentiation may occur under normoxia conditions for a period of 1-6 days. In some embodiments, the culturing of cells in the second phase of erythroid differentiation occurs under hypoxia conditions. The hypoxia conditions may comprise 2% O₂, or may comprise 2% O₂ and 5% CO₂.

In some embodiments, the culturing of cells in the second phase of erythroid differentiation occurs under hypoxia conditions for a period of 1-15 days, a period of 1-12 days, or for a period of 12 days. In some embodiments, an erythroid differentiation medium is switched to Iscove's Modified Dulbecco's Medium (IMDM) on day 12 of the second phase of erythroid differentiation, and wherein the cells are incubated under hypoxia conditions for at least 15 hours.

In some embodiments, the cells are fixed under hypoxia conditions. In some embodiments, the differentiated erythroid cells are stained with thiazole orange. In some embodiments, the imaging device is a flow cytometry device (e.g., an Amnis ImageStream device).

In some embodiments, the potency assay further comprises calculating the shape ratio for the fixed and stained erythroid cells, wherein the shape ratio is calculated as the minimum thickness of the cell divided by the length of the cell. In some embodiments, the Sickle Index value is calculated as the shape ratio divided by the area of each cell, and wherein the shape ratio is calculated as the minimum thickness of the cell divided by the length of the cell. In some embodiments, the percent of sickled erythroid cells is calculated by identifying the percent of erythroid cells in the population having a Sickle Index value less than 0.004.

In some embodiments, the potency assay further comprises analyzing untransduced cells in a second population of hematopoietic stem or progenitor cells from the subject with the imaging device. In some embodiments, the potency assay further comprises calculating the shape ratio for the untransduced cells, wherein the shape ratio is calculated as the minimum thickness of the cell divided by the length of the cell. In some embodiments, the potency assay further comprises calculating a Sickle Index value for the untransduced cells, wherein the Sickle Index value is calculated as the shape ratio divided by the area of each cell, and wherein the shape ratio is calculated as the minimum thickness of the cell divided by the length of the cell. In some embodiments, the potency assay further comprises calculating the percent of sickled untransduced cells, wherein the percent of sickled untransduced cells is calculated by identifying the percent of untransduced cells in the second cell sample having a Sickle Index value less than 0.004. In some embodiments, the potency assay further comprises calculating the relative potency of gene therapy treatment, wherein the relative potency is calculated as the percent sickled untransduced cells minus the percent sickled transduced cells divided by the percent sickled untransduced cells.

Also disclosed herein are methods for measuring relative potency of a drug product. The methods comprise calculating a Sickle Index value for a first population of hematopoietic stem or progenitor cells transduced with a lentiviral vector comprising a polynucleotide encoding a globin and for a second population of untransduced hematopoietic stem or progenitor cells, wherein the formula for calculating the Sickle Index value is:

$$Sickle Index = \frac{( minimum thickness length of each cell )}{area of each cell} ;$$

identifying the percent of sickled cells in a sample, wherein the cells are considered to be sickled if the Sickle Index value is less than 0.004; and calculating the relative potency of the drug product, wherein the formula for calculating relative potency is:

$$Relative Potency \% = \frac{( \% sickled untransduced - \% sickled transduced )}{\% sickled untransduced} ,$$

wherein the first population and the second population are obtained from a patient having sickle cell disease.

In some embodiments, the methods further comprises obtaining the hematopoietic stem or progenitor cells from the subject that has sickle cell disease. In some embodiments, the hematopoietic stem or progenitor cells comprise CD34⁺ cells, CD133⁺ cells, or CD34⁺CD38^{Lo}CD90⁺CD45RA⁻ cells. In some embodiments, the hematopoietic stem or progenitor cells comprise a pair of β-globin alleles selected from the group consisting of: β^{E}/β^{S}, β⁰/β^{S}, β^{C}/β^{S}, β⁺/β^{S} and β^{S}/β^{S. In some embodiments, the globin is a human β-globin, a human δ-globin, an anti-sickling globin, a human γ-globin, a human β}^{A-T87Q}-globin, a human β^{A-G16D/E22A/T87Q}-globin, or a human β^{A-T87Q/K95E/K120E}-globin protein. In some embodiments, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, or a derivative thereof.

In some embodiments, the Sickle Index value is calculated using a flow cytometry device (e.g., an Amnis ImageStream device). In some embodiments, the population of hematopoietic stem or progenitor cells transduced with the lentiviral vector are differentiated using a two-phase erythroid differentiation protocol before the Sickle Index value is calculated. In some embodiments, the second phase of the erythroid differentiation protocol occurs under hypoxia conditions. The hypoxia conditions may comprise 2% O₂. In some embodiments, the second phase of the erythroid differentiation protocol occurs for a period of 1 to 15 days, or for a period of 12 days. In some embodiments, the erythroid differentiated hematopoietic stem or progenitor cells are fixed under hypoxia and stained with thiazole orange.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a mutation in β-globin gene leads to abnormal RBC sickle shape under hypoxia/low-oxygen conditions. A morphology-based potency assay for sickle cell focuses on this conformational change that occurs under low oxygen conditions.

FIG. 2A-2D demonstrate a Sickle Index analysis of cells. A Sickle Index analysis is used to distinguish sickled cells from round cells by creating a mask that identifies the object or cell (FIG. 2A). The shape ratio, which is the measurement of the thinnest part of the cell divided by the length, is then calculated (FIG. 2B). To calculate the Sickle Index, the shape ratio value is then divided by the total area of the cell (FIG. 2C). The Sickle Index value is then compared between sickled cells and round cells. The Sickle Index value of a round non-sickled cell is higher, often significantly higher, than the Sickle Index for a sickled cell (FIG. 2D).

FIG. 3 demonstrates Sickle Index analysis on whole blood samples. A histogram is obtained showing the Sickle Index analysis of sickled red blood cells on the left (red) and the Sickle Index analysis of healthy whole red blood cells on the right (blue). The representative images for the sickled red blood cells are images of sickled cells. The representative images for the healthy whole blood cells are images of a classic donut shape. At the middle of the histogram there is some overlap of RBCs coming through on their side that are misidentified as sickled cells, or RBCs that are sickled into a circular shape and are therefore misidentified as healthy blood cells.

FIG. 4 demonstrates Sickle Index analysis on erythroid differentiated SCD CD34⁺ cells. The Sickle Index analysis on differentiated CD34 cells results in two distinct peaks. The left-most peak contains the very obviously sickled cells, and the right-most peak contains the round cells that look very much like red blood cells.

FIG. 5 shows that the Sickle Index assay is robust. The Sickle Index cutoff was changed from 0.004 to 0.005, but does not significantly impact assay results.

FIG. 6 shows % sickled cells obtained after erythroid differentiation culture of untransduced CD34⁺ cells or CD34⁺ cells transduced with an LVV encoding an anti-sickling β-globin cultured in normoxia (21% Oxygen) for the first phase of culture (days 1-6) compared to hypoxia (2% Oxygen) for the second phase of culture (days 7-21). Hypoxic conditions give larger relative differences in % sickled cells between transduced and untransduced cells as compared to normoxic conditions.

FIG. 7 shows that Day 18 of erythroid differentiation shows the maximum sickling of untransduced CD34⁺ cells or CD34⁺ cells transduced with an LVV encoding an anti-sickling β-globin with peak relative difference to mock. The day of analysis is the day that cells were resuspended in serum-free media at a standard density and volume. Cells were cultured overnight in hypoxia and then fixed with glutaraldehyde immediately.

FIG. 8 shows linearity of the Sickle Index method using mixtures of red blood cells from a healthy donor and sickle subject. Linearity was assessed by diluting SCD RBCs with healthy subject RBCs.

FIG. 9 shows dilutional linearity of the Sickle Index method using Day 18 erythroid differentiated cells. Untransduced sickled CD34⁺ and source-matched CD34⁺ cells transduced with an LVV encoding an anti-sickling β-globin (VCN=4.0 copies per diploid genome (c/dg)) were differentiated in parallel using erythroid differentiation culture. Untransduced cells were mixed with transduced cells on day 18 of erythroid differentiation culture and acquired in triplicate.

FIG. 10 shows sample stability in the Sickle Index method. Three cell lots were tested for sample stability and are represented on the X-axis. Untransduced CD34⁺ cells and CD34⁺ cells transduced with an anti-sickling β-globin from each sample was fixed and analyzed by the Sickle Index method on the day of fixation and again 90 days later.

FIG. 11 shows non-specific activity in the Sickle Index method that results from stimulation culture and LVV transduction. Sickled CD34⁺ cells (SCD) and normal CD34⁺ cells were stimulated and transduced with a LVV. The VCN of CD34⁺ cells transduced with the LVV is shown. Potency, calculated as percent decrease in anti-CD36-PE MFI relative to the non-stimulated, untransduced control is indicated in black bars in cases where it exceeded 10%.

FIG. 12 shows non-specific activity in the Sickle Index method that results from an additional 24 hours of stimulation culture. Sickled CD34⁺ cells were stimulated either for 48 or 72 hours prior to erythroid differentiation and the resulting cells were compared by the Sickle Index method. The highest observed non-specific potency was a 15.8% decrease in sickled cells (middle panel).

FIG. 13 shows potency of drug product manufactured from CD34⁺ sickled cells calculated using the Sickle Index method. Drug products (Table 3) and subject- and batch-matched stimulated untransduced control CD34⁺ cells were tested for potency by the Sickle Index method. Potency, calculated for each pair of samples as the reduction in sickled cells, is indicated with black bars. Results are arranged by increasing VCN.

FIGS. 14A-14B demonstrate correlation between VCN, % LVV, and potency by Sickle Index. Data from all tested drug products (DP) (Table 3) is plotted. FIG. 14A shows VCN values are from the 14 day pooled colony assay. Dashed line indicates maximum non-specific background (FIG. 12). FIG. 14B shows % LVV values that are from single cell PCR assay. Dashed lines show that at 33.0% LVV⁺ cells (equivalent to our VCN spec of 0.8 c/dg) an anti-sickling potency of 27.1% was expected.

FIG. 15 shows mixes of healthy and sickle subject blood controls for system suitability. The system suitability results from qualification experiments are shown.

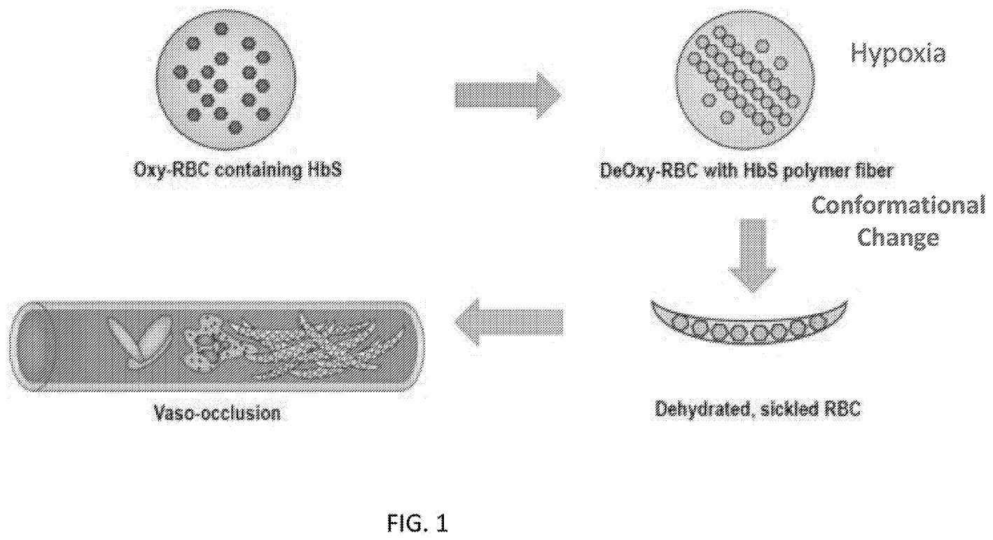

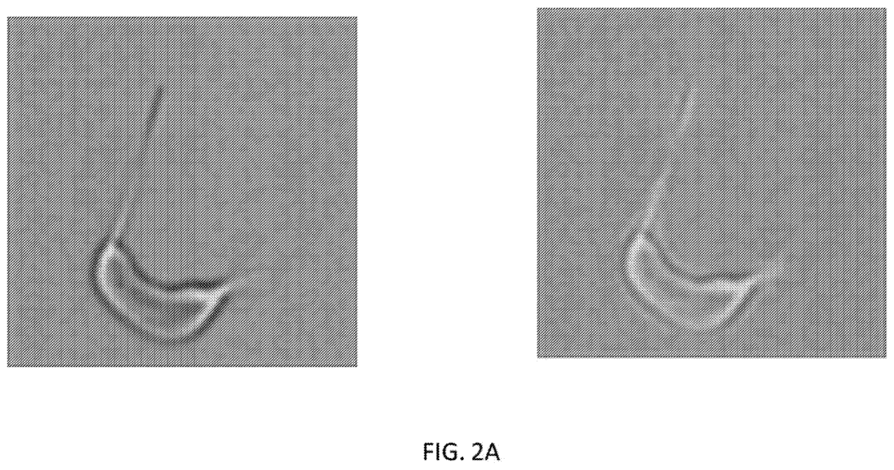

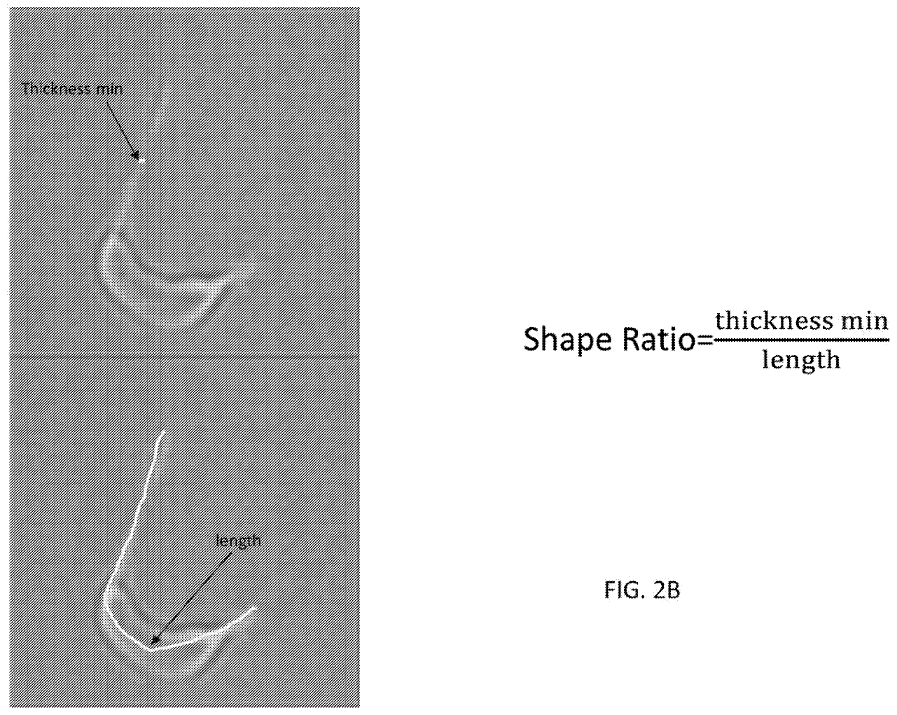

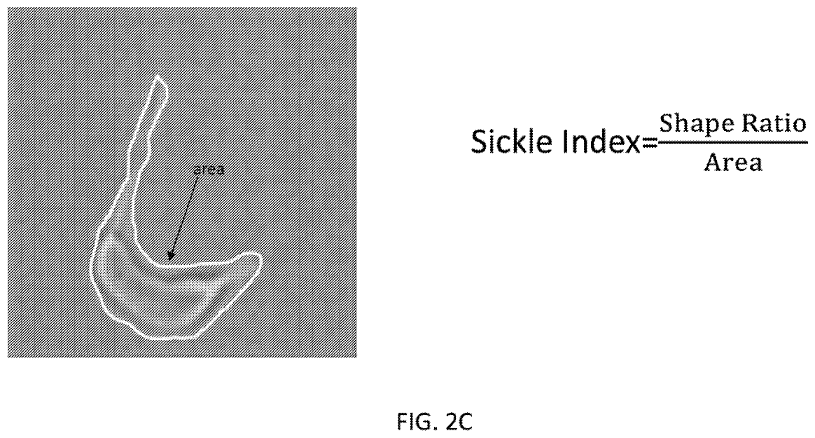

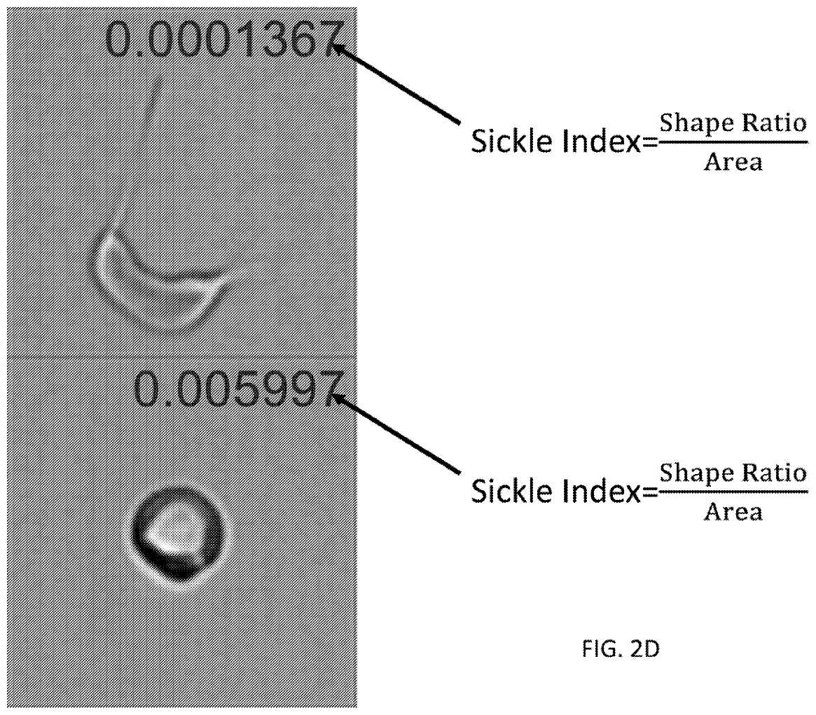

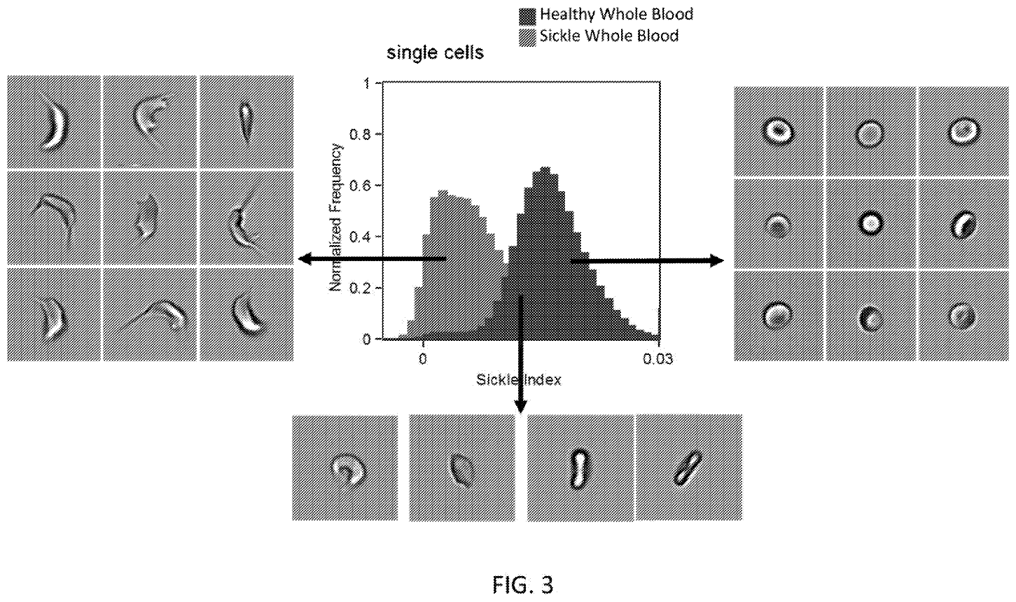

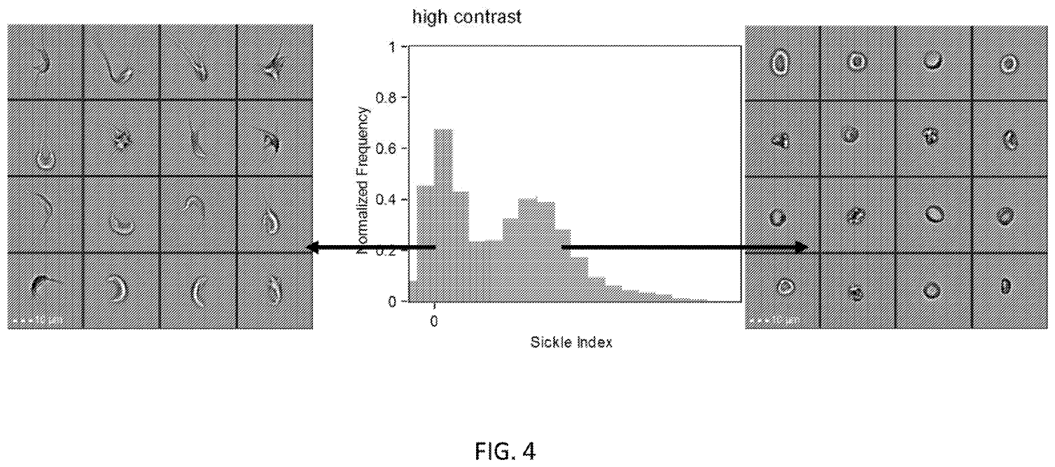

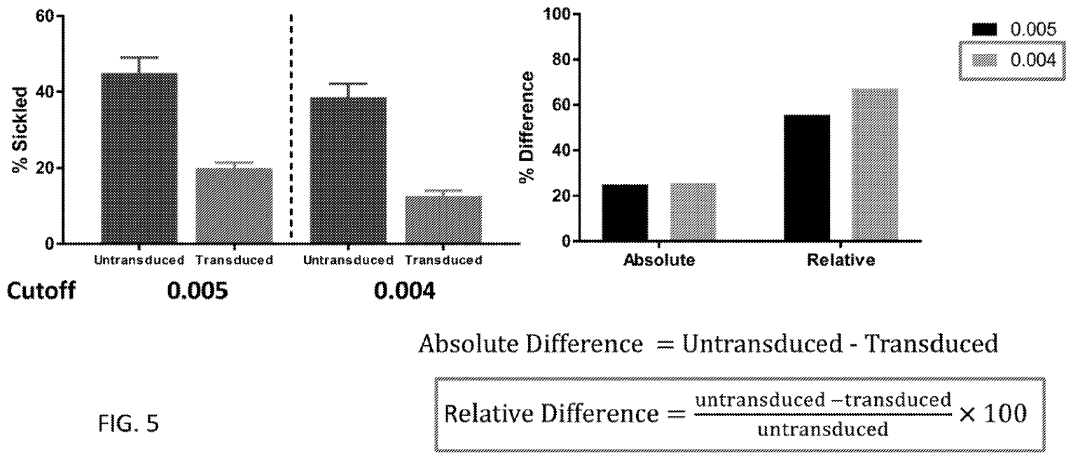

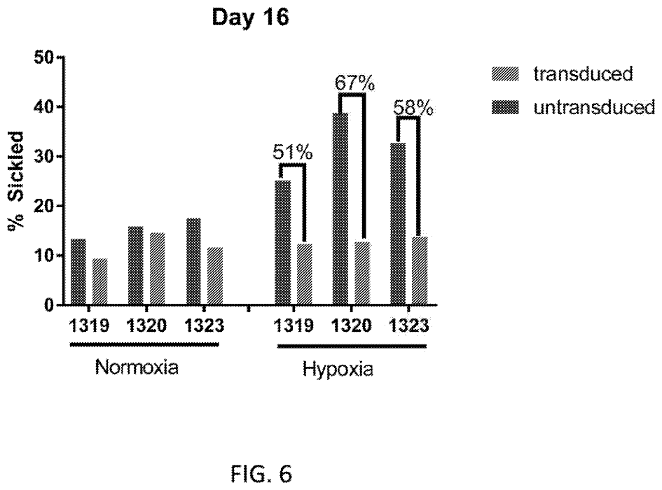

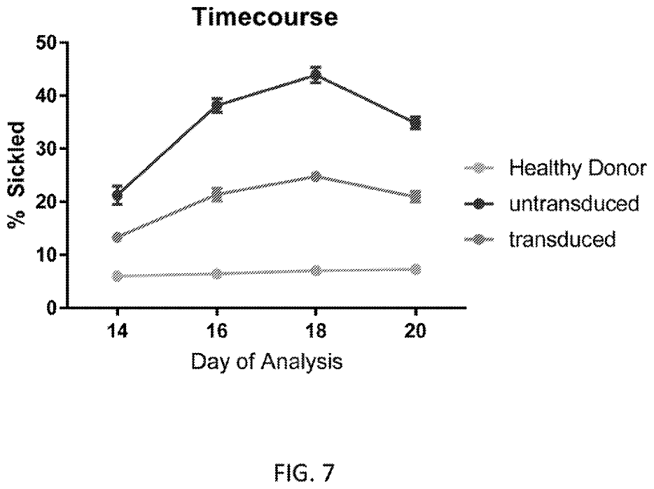

### DETAILED DESCRIPTION OF THE INVENTION

A robust and objective assay that can quantify the hypoxia-induced morphological change of SCD RBCs differentiated from CD34⁺ hematopoietic stem and progenitor cells (HSPCs) is described herein. Moreover, this assay can be used to assess the relative level of correction of this morphological change in RBCs differentiated from SCD HSPCs transduced with a lentiviral vector (LVV) comprising a polynucleotide encoding therapeutic globin, and in some preferred embodiments, an anti-sickling β-globin. Disclosed herein are potency assays for a gene therapy treatment for sickle cell disease (SCD). Also disclosed herein are methods for measuring relative potency of a drug product.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements. The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. As used herein, the terms “include” and “comprise” are used synonymously. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., lentiviral vectors.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term “viral vector” may refer either to a virus or viral particle capable of transferring a nucleic acid into a cell or to the transferred nucleic acid itself. Viral vectors and transfer plasmids contain structural and/or functional genetic elements that are primarily derived from a virus. The term “lentiviral vector” refers to a retroviral vector or plasmid containing structural and functional genetic elements, or portions thereof, including LTRs that are primarily derived from a lentivirus. The terms “lentiviral vector” and “lentiviral expression vector” may be used to refer to lentiviral transfer plasmids and/or infectious lentiviral particles in particular embodiments. Where reference is made herein to elements such as cloning sites, promoters, regulatory elements, heterologous nucleic acids, etc., it is to be understood that the sequences of these elements are present in RNA form in the lentiviral particles contemplated herein and are present in DNA form in the DNA plasmids contemplated herein.

“Transfection” refers to the process of introducing naked DNA into cells by non-viral methods.

“Infection” refers to the process of introducing foreign DNA into cells using a viral vector. “Transduction” refers to the introduction of foreign DNA into a cell's genome using a viral vector.

“Vector copy number” or “VCN” refers to the number of copies of a vector, or portion thereof, in a cell's genome. The average VCN may be determined from a population of cells or from individual cell colonies.

“Transduction efficiency” refers to the percentage of cells transduced with at least one copy of a vector.

The term “globin” as used herein refers to proteins or protein subunits that are capable of covalently or noncovalently binding a heme moiety, and can therefore transport or store oxygen. Subunits of vertebrate and invertebrate hemoglobins, vertebrate and invertebrate myoglobins or mutants thereof are included by the term globin. The term excludes hemocyanins. Examples of globins include α-globin or variants thereof, β-globin or variants thereof, γ-globin or variants thereof, and δ-globin or variants thereof.

Additional definitions are set forth throughout this disclosure.

In the following description, certain specific details are set forth in order to provide a thorough understanding of various illustrative embodiments of the invention contemplated herein. However, one skilled in the art will understand that particular illustrative embodiments may be practiced without these details.

### Potency Assays

Disclosed herein are potency assays for a gene therapy treatment for sickle cell disease (SCD). In some embodiments, a potency assay comprises transducing a population of hematopoietic stem or progenitor cells from a subject that has sickle cell disease with a lentiviral vector comprising a polynucleotide encoding a globin; performing two-phase erythroid differentiation of the population of hematopoietic stem or progenitor cells comprising culturing the hematopoietic stem or progenitor cells under hypoxia during erythroid differentiation; fixing and staining the differentiated erythroid cells; analyzing the fixed and stained erythroid cells with an imaging device; calculating a Sickle Index value for the analyzed erythroid cells; and calculating the percent of sickled erythroid cells in the population, wherein the potency of the gene therapy treatment is the proportion of sickled cells in the population relative to an untransduced control.

In some embodiments, a potency assay for a gene therapy treatment for sickle cell disease (SCD) comprises performing two-phase erythroid differentiation of a population of hematopoietic stem or progenitor cells from a subject that has sickle cell disease, wherein the population of hematopoietic stem or progenitor cells are transduced with a lentiviral vector comprising a polynucleotide encoding a globin; culturing the population of hematopoietic stem or progenitor cells under hypoxia during erythroid differentiation; fixing and staining the differentiated erythroid cells; analyzing the fixed and stained erythroid cells with an imaging device; calculating a Sickle Index value for the analyzed erythroid cells; and calculating the percent of sickled erythroid cells in the sample, wherein the potency of the gene therapy treatment is the proportion of sickled erythroid cells in the population of hematopoietic stem or progenitor cells of step a) relative to a population of untransduced hematopoietic stem or progenitor cells.

In particular aspects, the method comprises obtaining a population or sample of hematopoietic stem or progenitor cells from a subject that has sickle cell disease. Suitable methods for obtaining hematopoietic stem or progenitor cells from a subject include apheresis.

In some aspects, hematopoietic stem or progenitor cells are selected from the group consisting of CD34⁺ cells, CD133⁺ cells, CD34⁺CD38^{Lo}CD90⁺CD45RA⁻ cells, and combinations thereof. In certain aspects, the hematopoietic stem or progenitor cells include CD34⁺ cells. In certain aspects, the hematopoietic stem or progenitor cells include CD133⁺ cells. In certain aspects, the hematopoietic stem or progenitor cells include CD34⁺CD38^{Lo}CD90⁺CD45RAf⁻ cells.

In some aspects, the hematopoietic stem or progenitor cells comprise a pair of β-globin alleles selected from the group consisting of β^{E}/β^{S}, β⁰/β^{S}, β^{C}/β^{S}, β⁺/β^{S} and β^{S}/β^{S}. In certain aspects, the hematopoietic stem or progenitor cells comprise a pair of β-globin alleles that are β^{E}/β^{S}. In certain aspects, the hematopoietic stem or progenitor cells comprise a pair of β-globin alleles that are β⁰/β^{S}. In certain aspects, the hematopoietic stem or progenitor cells comprise a pair of β-globin alleles that are β^{C}/β^{S}. In certain aspects, the hematopoietic stem or progenitor cells comprise a pair of β-globin alleles that are β⁺/β^{S}. In certain aspects, the hematopoietic stem or progenitor cells comprise a pair of β-globin alleles that are β^{S}/β^{S}.

In some embodiments, the hematopoietic stem or progenitor cells are transduced with a vector (e.g., a lentiviral vector) comprising a polynucleotide encoding a globin. In some aspects, the globin is a human β-globin, a human S-globin, an anti-sickling globin, a human γ-globin, a human β^{A-T87Q}-globin, a human β^{A-G16D/E22A/T87Q}-globin, or a human β^{A-T87Q/K95E/K120E}-globin protein. In certain aspects, the globin is a human β-globin protein. In certain aspects, the globin is a human δ-globin protein. In certain aspects, the globin is an anti-sickling globin protein. In certain aspects, the globin is a human γ-globin protein. In certain aspects the globin is a human β^{A-T87Q}-globin protein. In certain aspects, the globin is a human β^{A-G16D/E22A/T87Q}-globin protein. In certain aspects, the globin is a human β^{A-T87Q/K95E/K120E}-globin protein.

In some embodiments the vector is a lentiviral vector. In some aspects, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, or a derivative thereof. In some aspects, the lentiviral vector is an AnkT9W vector or a derivative thereof. In some aspects, the lentiviral vector is a T9Ank2W vector or a derivative thereof. In some aspects, the lentiviral vector is a TNS9 vector or a derivative thereof. In some aspects, the lentiviral vector is a TNS9.3 vector or a derivative thereof. In some aspects, the lentiviral vector is a TNS9.3.55 vector or a derivative thereof. In some aspects, the lentiviral vector is a lentiglobin HPV569 vector or a derivative thereof. In some aspects, the lentiviral vector is a lentiglobin BB305 vector or a derivative thereof. In some aspects, the lentiviral vector is a BG-1 vector or a derivative thereof. In some aspects, the lentiviral vector is a BGM-1 vector or a derivative thereof. In some aspects, the lentiviral vector is a d432βAγ vector or a derivative thereof. In some aspects, the lentiviral vector is a mLARβΔγV5 vector or a derivative thereof. In some aspects, the lentiviral vector is a GLOBE vector or a derivative thereof. In some aspects, the lentiviral vector is a G-GLOBE vector or a derivative thereof. In some aspects, the lentiviral vector is a βAS3-FB vector or a derivative thereof. In some aspects, the lentiviral vector is a V5 vector or a derivative thereof. In some aspects, the lentiviral vector is a V5m3 vector or a derivative thereof. In some aspects, the lentiviral vector is a V5m3-400 vector or a derivative thereof. In some aspects, the lentiviral vector is a G9 vector or a derivative thereof.

In some embodiments, erythroid differentiation is performed in HiF erythroid differentiation media (Iscove's Modified Dulbecco's Medium (IMDM), 20% FBS, 20 ng/mL rhSCF, 1 ng/mL rhIL-3, 2 Units/mL rhEPO). HiF erythroid differentiation media may be used for the first and/or second phase of erythroid differentiation. In some aspects, HiF erythroid differentiation media is used for the entire first and/or second phase of erythroid differentiation, or in other aspects, HiF erythroid differentiation media is used for a portion of the first and/or second phase of erythroid differentiation. In some aspects, erythroid differentiation medium is switched to a second differentiation medium (IMDM, 20% FBS, 0.2 mg/mL hApo, 2 Units/mL rhEPO) at some point during the second phase of erythroid differentiation. For example, on day 18 of the differentiation protocol (i.e., day 11 of the second phase of erythroid differentiation), the differentiation medium is switched from HiF to a second differentiation medium. In some aspects, the cells are incubated in a second differentiation medium under hypoxia conditions for at least 10, 12, 15, or 18 hours. In certain aspects, the cells are incubated in a second differentiation medium under hypoxia conditions for at least 15 hours.

In some aspects, two-phase erythroid differentiation of the transduced hematopoietic stem or progenitor cells occurs for a period of at least 15, at least 16, at least 17, at least 18, at least 19, at least 20, at least 21, at least 22, at least 23, at least 24, or at least 25 days. In certain aspects, the first phase of erythroid differentiation occurs under normoxia or normoxic conditions (e.g., 21% Oxygen) for a period of 1 to 7 days. In certain aspects, the second phase of erythroid differentiation occurs under hypoxia or hypoxic conditions (e.g., 2% Oxygen) for a period of 5 to 25 days. In some embodiments, the first phase of erythroid differentiation occurs from day 1 to day 6 and the second phase of erythroid differentiation occurs from day 7 to day 21.

In some embodiments, the culturing of cells in the second phase of erythroid differentiation occurs under hypoxia. In some aspects, the hypoxic conditions comprise 0.01% to 5% 02. In certain aspects, the hypoxic conditions comprise 1%, 1.25%, 1.5%, 1.75%, 2%, 2.25%, 2.5%, 2.75%, or 3% 02. In some embodiments, the hypoxic conditions comprise 2% 02. In some aspects, the hypoxic conditions comprise 5% CO₂ and 2% 02. The culturing of cells in the second phase under hypoxia may occur for a period of 1 to 15 days, 5 to 13 days, or 8 to 12 days. In certain embodiments the culturing of cells in the second phase under hypoxia occurs for 12 days. In some aspects, the second phase of erythroid differentiation is performed in a hypoxia chamber (e.g., Billups Hypoxia Chamber) or a glovebox (e.g., Hypoxygen H35 glovebox). In some aspects, the second phase of erythroid differentiation is begun in a hypoxia chamber and is then completed in a glovebox.

In some embodiments, the differentiated erythroid cells are resuspended in serum-free media (e.g., IMDM) at a standard density and volume. The cells may be cultured overnight under hypoxic conditions in the serum-free media and then fixed under hypoxic conditions. In some aspects, the cells are cultured overnight in a glovebox (e.g., Hypoxygen H35 glovebox). In some aspects, the cells are fixed with glutaraldehyde. In certain aspects, the cells are fixed with glutaraldehyde immediately after overnight culture in hypoxic conditions. In some aspects, the fixed cells are stained. The fixed cells may be stained with thiazole orange.

The fixed and stained differentiated erythroid cells may be analyzed with an imaging device, such as a flow cytometry device (e.g., an Amnis ImageStream device). In some aspects, the imaging device calculates the area of each cell. In some aspects, the imaging device calculates a shape ratio for the fixed and stained cells. The shape ratio may be calculated as the minimum thickness of the cell divided by the length of the cell. In certain aspects, a Sickle Index value is calculated for the analyzed differentiated erythroid cells. The Sickle Index value may be calculated as the shape ratio divided by the area of each cell.

In some embodiments, a population of untransduced hematopoietic stem or progenitor cells (e.g., from the subject) are also analyzed with an imaging device, such as a flow cytometry device (e.g., an Amnis ImageStream device). In some aspects, the imaging device calculates a shape ratio and/or the area for the untransduced cells. In certain aspects, a Sickle Index value is calculated for the analyzed untransduced cells.

The calculated Sickle Index value may be used to identify the percent of sickled cells (e.g., sickled erythroid cells) in a sample. For example, RBCs are considered to be sickled if the Sickle Index value is less than 0.004. The percent of sickled erythroid cells in a sample may be calculated by determining the percentage of cells having a Sickle Index value of less than 0.004.

In some embodiments, the relative potency of a gene therapy treatment for sickle cell disease is calculated. In particular embodiments, the relative potency is calculated as the percent sickled untransduced cells minus the percent sickled transduced cells (e.g., erythroid cells, RBCs) divided by the percent sickled untransduced cells. The potency of a gene therapy treatment for sickle cell disease may be calculated as the proportion of sickled cells in a population or cell sample relative to the proportion of untransduced cells.

### Methods for Measuring Potency of a Drug Product

Also disclosed herein are methods for measuring relative potency of a drug product for treating sickle cell disease. In some aspects, the methods comprise calculating a Sickle Index value for a first population of hematopoietic stem or progenitor cells transduced with a lentiviral vector comprising a polynucleotide encoding a globin and for a second population of untransduced hematopoietic stem or progenitor cells, wherein the formula for calculating the Sickle Index is:

$$Sickle Index = \frac{( minimum thickness length of each cell )}{area of each cell} ;$$

and identifying the percent of sickled cells in a sample, wherein the cells are considered to be sickled if the Sickle Index value is less than 0.004; and calculating the relative potency of the drug product, wherein the formula for calculating relative potency is:

$$Relative Potency \% = \frac{( \% sickled untransduced - \% sickled transduced )}{\% sickled untransduced} ,$$

wherein the cells (e.g., the first population and the second population) are obtained from a patient having sickle cell disease.

In some embodiments, the Sickle Index value is calculated using a flow cytometry device (e.g., an Amnis ImageStream device).

In some embodiments, the population of hematopoietic stem or progenitor cells transduced with the lentiviral vector are differentiated using a two-phase erythroid differentiation protocol before the Sickle Index value is calculated. In some aspects, the erythroid differentiation protocol occurs over a period of 21 to 25 days. In some aspects, the period of days 1-6 is the first phase of the differentiation protocol, and occurs under normoxic conditions. In some aspects, the period of days 7-21 of the differentiation protocol is the second phase of the protocol, and occurs under hypoxic conditions. In some embodiments hypoxic conditions comprise 2% O₂ and 5% Co₂. In some embodiments the erythroid differentiated hematopoietic stem or progenitor cells are fixed under hypoxia conditions, and then stained with thiazole orange.

All publications, patent applications, and issued patents cited in this specification are herein incorporated by reference as if each individual publication, patent application, or issued patent were specifically and individually indicated to be incorporated by reference.

The following examples are provided by way of illustration only and not by way of limitation. Those of skill in the art will readily recognize a variety of noncritical parameters that could be changed or modified in particular embodiments to yield essentially similar results.

### Exemplification

### SUMMARY

A robust and objective assay that can quantify the hypoxia-induced morphological change of SCD RBCs differentiated from CD34⁺ hematopoietic stem and progenitor cells (HSPCs) is described herein. Moreover, this assay can be used to assess the relative level of correction of this morphological change in RBCs differentiated from SCD HSPCs transduced with a lentiviral vector (LVV) encoding a globin including, but not limited to, β-globin, γ-globin, or an anti-sickling β-globin (e.g., β-globin^{AT87Q}). Transduced and cell lot-matched untransduced control HSPCs were cultured in a two-phase erythroid differentiation protocol to generate RBCs, with the second phase of culture performed in 2% oxygen. Cells were fixed, stained with thiazole orange, and cell images collected on the Amnis ImageStream imaging flow cytometer. Fixed erythroid differentiated cells were found to be stable for up to 3 months. Images of the erythroid differentiated RBCs were then analyzed using a stringent gating strategy to determine the proportion of sickled cells in the cell population and to quantify the relative amelioration of disease phenotype (sickling) in RBCs derived from transduced cells.

Assessment of assay readout precision spanning two operators, two sampling time points, and two instruments resulted in a 5.8% CV, indicating reliable performance of the method and ImageStream analysis. Assessment of overall assay precision spanning six cell culture runs with two operators and three to six replicates of test article per cell culture run resulted in a 4.0% CV for untransduced cells and 9.2% CV for cells transduced with LVV encoding an anti-sickling β-globin, e.g., LentiGlobin BB305 LVV. Transduction with LentiGlobin BB305 LVV resulted in a decrease of the proportion of sickled cells compared to the untransduced controls across cells from multiple SCD subjects, and the reduction in sickling was found to be specific to transduction with LVVs that lead to increased expression of anti-sickling β-globin. This relative decrease correlated with vector copy number (VCN), the percentage of transduced cells, and the amount of protein expressed.

This assay is robust, precise, and suitable for the in vitro characterization of the anti-sickling properties of LVV encoding an anti-sickling β-globin in SCD CD34⁺ cells. Moreover, these data demonstrate the marked reduction in the sickle RBC phenotype in vitro driven by transduction with LVV encoding an anti-sickling β-globin.

### Results

A drug product potency assay (also referred to herein as the Sickle Index method) was developed and assessed for accuracy, dilutional linearity, range, and specificity. The specificity of the assay to detect transgene activity was assessed. The specificity is the ability of the potency method to obtain positive results from samples containing the analyte and negative results from samples that do not contain the analyte. The method was used to measure potency of GMP drug products manufactured from SCD HSPCs with known anti-sickling β-globin^{AT87Q} expression levels. Once this had been evaluated, repeatability and intermediate precision studies were evaluated with the expected resulting % CV to be at or below 25%.

Accuracy, Linearity, Range, and Specificity Accuracy, specificity, range, and linearity were assessed for the Sickle Index method. In one experiment accuracy, linearity, range, and specificity was assessed using red blood cells with a 19-day erythroid differentiation protocol. In a second experiment, linearity was assessed using day 19 erythroid differentiated cells.

Accuracy, Linearity, Range, and Specificity Using Red Blood Cells

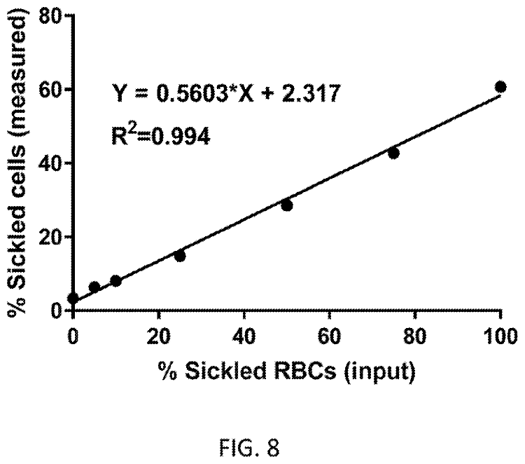

For this study, red blood cells from a healthy donor and a sickle subject were first incubated at 2% O₂ overnight to induce sickling and fixed with 0.15% glutaraldehyde. The cells were then counted and mixed at ratios of 0%, 5%, 10%, 25%, 50%, 75%, and 100% sickled RBCs with each mixture being acquired in triplicate on the Amnis ImageStream and analyzed using Amnis IDEAS. These known mixes were used to determine the assay range, accuracy, and specificity (Table 1). The Sickle Index method was not able to detect 100% sickle subject blood as 100% sickled, and healthy donor blood as 0% sickled stems (FIG. 8) due to the stringency of the gate to detect sickled cells and filter out cells that may look sickled, such as RBCs being imaged at different angles. The mixtures of healthy and sickle blood were highly linear (R2=0.994), thus, the method is appropriate for a potency assay where potency is calculated as a relative decrease in the percentage of sickled cells compared to a subject- and batch-matched control due to the high linearity of the assay.

TABLE 1
Linearity of the Sickle Index method using mixtures of red blood
cells from a sickle subject and healthy donor.

| | Replicate 1 | Replicate 2 | Replicate 3 | Average % | | |
|---|---|---|---|---|---|---|
| | % Sickled | % Sickled | % Sickled | Sickled | | |
| | RBCs by | RBCs by | RBCs by | RBCs by | | Percent |
| | Sickle | Sickle | Sickle | Sickle | Standard | CV |
| Sample | Index | Index | Index | Index | Deviation | (Precision) |
| 0% | 4.1 | 3.7 | 2.4 | 3.4 | 0.9 | 26.1% |
| Sickled | | | | | | |
| RBCs | | | | | | |
| 5% | 6.7 | 6.2 | 6.1 | 6.3 | 0.3 | 5.1% |
| Sickled | | | | | | |
| RBCs | | | | | | |
| 10% | 7.9 | 8.1 | 8.4 | 8.1 | 0.3 | 3.1% |
| Sickled | | | | | | |
| RBCs | | | | | | |
| 25% | 14.4 | 15.3 | 15.0 | 14.9 | 0.5 | 3.1% |
| Sickled | | | | | | |
| RBCs | | | | | | |
| 50% | 27.6 | 29.3 | 28.7 | 28.5 | 0.9 | 3.0% |
| Sickled | | | | | | |
| RBCs | | | | | | |
| 75% | 42.1 | 42.2 | 43.9 | 42.7 | 1.0 | 2.4% |
| Sickled | | | | | | |
| RBCs | | | | | | |
| 100% | 61.0 | 60.3 | 61.0 | 60.8 | 0.4 | 0.7% |
| Sickled | | | | | | |
| RBCs | | | | | | |

Accuracy, Linearity, Range, and Specificity Using Erythroid Differentiated Cells

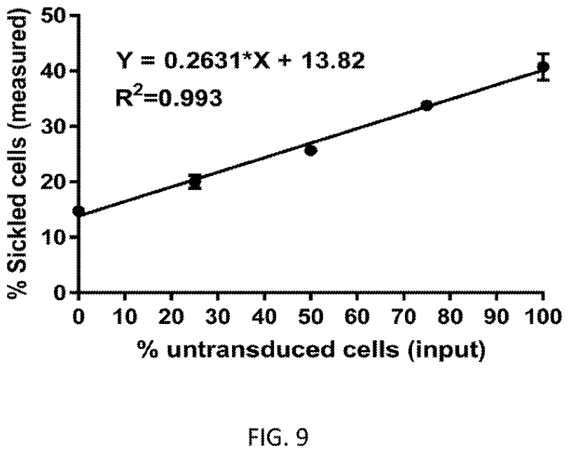

Because the Sickle Index method uses erythroid differentiated cells and not cells from peripheral blood, the effect of matrix interference from other cells and molecules generated during the 19-day erythroid differentiation was evaluated. SCD CD34⁺ cells were transduced with LentiGlobin BB305 LVV (VCN=4.0 c/dg) and erythroid differentiated for 7 days in normoxic culture and 12 days in culture at 2% O₂, along with post-stimulated untransduced SCD CD34⁺ cells from the same source. Erythroid differentiated cells resulting from transduced and untransduced CD34⁺ cells were mixed on day 19 of culture at different ratios to mimic the peripheral blood mixing experiment detailed in FIG. 8 and acquired in triplicate on the Amnis ImageStream and analyzed using Amnis IDEAS. The percentage of sickled cells was found to decrease linearly as the number of transduced cells in the mixture increased (R2=0.993) (FIG. 9). The maximum observed standard deviation was 2.4% sickled cells (Table 2) and therefore, the minimum resolvable difference between samples is 7.2% sickled cells (three times the highest standard deviation). This experiment indicated that the range observed with the mixtures of healthy and SCD in-vitro derived RBCs was within the range of healthy and SCD peripheral blood controls (FIG. 8), and because the method is linear, it is appropriate for a potency assay that uses a subject and batched matched control as a comparator.

TABLE 2
Dilutional linearity of the Sickle Index method using Day 19
erythroid differentiated cells

| | Replicate 1 | Replicate 2 | Replicate 3 | Average % | | |
|---|---|---|---|---|---|---|
| | % Sickled | % Sickled | % Sickled | Sickled | | |
| | RBCs by | RBCs by | RBCs by | RBCs by | | Percent |
| | Sickle | Sickle | Sickle | Sickle | Standard | CV |
| Sample | Index | Index | Index | Index | Deviation | (Precision) |
| 0% | 14.4 | 15.2 | 14.5 | 14.7 | 0.4 | 3.0% |
| Sickled | | | | | | |
| RBCs | | | | | | |
| 25% | 21.3 | 19.8 | 19.0 | 20.0 | 1.2 | 5.9% |
| Sickled | | | | | | |
| RBCs | | | | | | |
| 50% | 25.2 | 25.1 | 26.6 | 25.6 | 0.8 | 3.3% |
| Sickled | | | | | | |
| RBCs | | | | | | |
| 75% | 32.9 | 34.7 | 33.7 | 33.8 | 0.9 | 2.6% |
| Sickled | | | | | | |
| RBCs | | | | | | |
| 100% | 38.0 | 42.5 | 41.7 | 40.7 | 2.4 | 5.8% |
| Sickled | | | | | | |
| RBCs | | | | | | |

Stability of Readout

In the event that potency cannot be assessed immediately, it is important that the readout remain stable over time to reduce the risk of assay failure.

To assess the stability of the Sickle Index method, erythroid differentiated cells derived from transduced SCD CD34⁺ cells and matched, stimulated, untransduced controls from three cell lots were cultured at 2% O₂, fixed with 0.15% glutaraldehyde, and analyzed to determine % sickled cells. The cells were then stored at 4° C. for 90 days prior to re-analysis by the Sickle Index method.

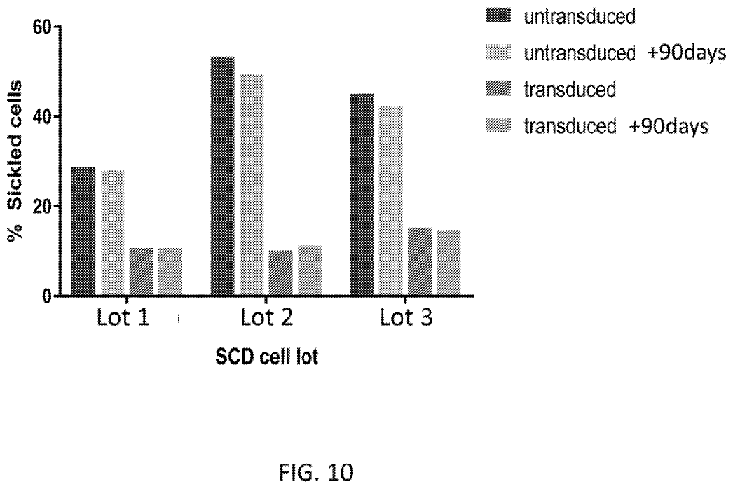

The average difference in antisickling potency between all re-analyzed replicates was 2.2% with the maximum difference in anti-sickling potency between two replicates being 3.7% (FIG. 10). Therefore, the Sickle Index method's potency measurement is stable for at least 90 days.

Assay Specificity to Transgene Activity

A potency method must obtain positive results from samples containing the analyte and negative results from samples that do not contain the analyte. Because potency is a relative measurement between transduced and untransduced cells, the relevant “analyte” is the activity of the therapeutic transgene in correcting SCD-associated dyserythropoiesis and conformational change. The Sickle Index method does not detect potency in CD34⁺ cells from healthy donors transduced with LentiGlobin BB305 LVV because healthy donor cells have no SCD-associated dyserythropoiesis or sickle hemoglobin polymerization. Mock transductions, or transductions without the use of LentiGlobin BB305 LVV, of CD34⁺ cells are also not expected to show potency. Transduction with an LVV that contains only the long terminal repeat sequences for integration into the genome and the PsiGag sequence for VCN analysis but lacks the beta globin promoter and transgene should also lack potency.

During drug product (DP) manufacturing, CD34⁺ cells were prestimulated with cytokine containing media for 44-48 hours prior to transduction. Although erythroid differentiated cells from stimulated, untransduced CD34⁺ cells were intended to be used as the untransduced comparator for the potency calculation, in this study erythroid differentiated cells from non-stimulated, untransduced CD34⁺ cells were also tested as a comparator to assess the impact of time in the stimulation cell culture to produce non-specific potency.

Non-Specific Activity from Stimulation Cell Culture and LVV Transduction

To test for non-specific potency, CD34⁺ cells from 3 SCD cell lots and 3 healthy donor cell lots were stimulated with cytokine media for 48 hours and then erythroid differentiated, and evaluated for potency by the Sickle Index method after 19 days in culture. Non-stimulated, untransduced CD34⁺ cells from the same cell lots were differentiated in parallel as a comparator to assess the effect of time in stimulation culture on potency.

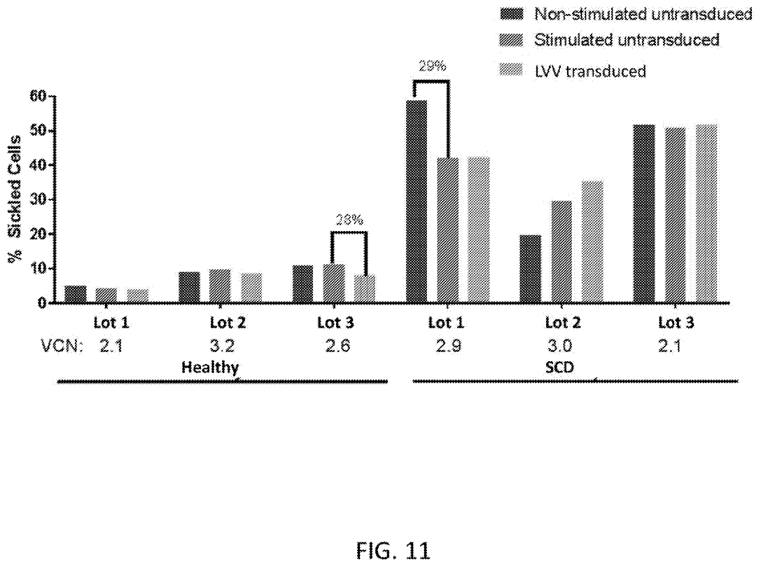

Out of the six tested cell lots, only one showed non-specific potency higher than the expected 10% (cell lot 4, FIG. 11, 29% reduction in sickled cells from nonstimulated, untransduced control). The other 5 lots showed the expected non-specific potency <10%. When comparing the anti-sickling activity of the LVV lacking the therapeutic globin to the stimulated, untransduced control, one healthy donor (cell lot 3, FIG. 11) had a 28% relative reduction in sickled cells, although the absolute magnitude of this change is only 3.2% (from 11.4% to 8.2%) and this difference is not resolvable according to the minimum resolvable difference of 7.2% sickled cells previously calculated.

Non-Specific Activity from Additional 24 Hours of Stimulation Culture

Because substantial (>10%) non-specific potency was observed when comparing stimulated, untransduced samples to non-stimulated, untransduced controls, the non-stimulated untransduced cells collected during LentiGlobin BB305 DP manufacturing are not a suitable comparator for the potency assay. Stimulated, untransduced cells were also collected on the second day of DP manufacturing, and DP samples would experience only an additional 20-24 hours of stimulation culture, thereby lessening the non-specific potency observed from stimulation culture.

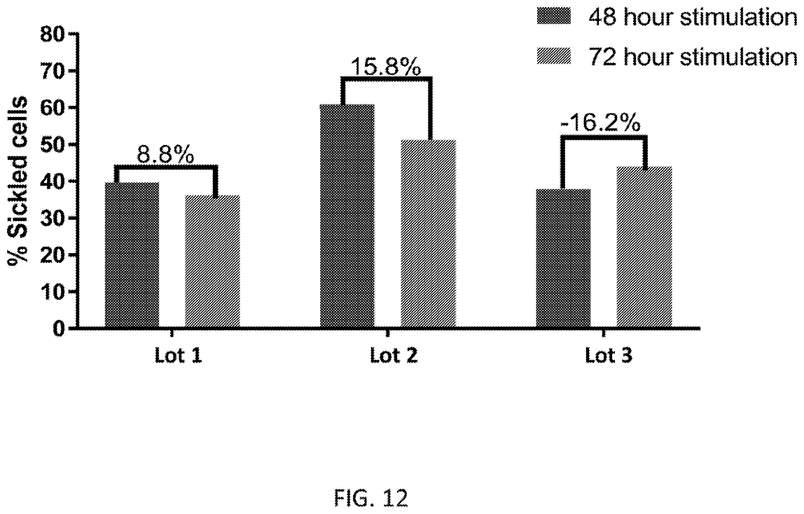

To measure the impact of the additional 24 hours of stimulation culture on potency, three different SCD CD34⁺ cell lots were thawed and cultured in stimulation media (SCGM supplemented with FLT3, SCF, TPO). A second group of the same three cell lots was thawed 24 hours later and cultured in stimulation media. After 72 hours of stimulation culture for the first group and 48 hours of stimulation culture for the second group, cells were transferred to erythroid differentiation cell culture. Potency was evaluated by the Sickle Index method after 7 days in normoxic culture and 12 days in culture at 2% O₂. Potency was calculated as percent change from cells in 48 hour stimulation culture to cells in 72 hour stimulation culture. The highest observed non-specific potency for the Sickle Index method was 15.8% (FIG. 12).

In conclusion, although stimulation cell culture used for DP manufacturing process does introduce some non-specific potency, the non-specific potency can be reduced when using a stimulated, untransduced control as a subject- and batch-matched comparator sample. The levels of nonspecific potency are useful for determination of minimum potency specification as the observed potency of the DP must be greater than the maximum non-specific potency shown in FIG. 12.

Proof of Concept Studies

Before continuing with qualification for the potency method, it was important to demonstrate the capability of the method at measuring potency in drug product (DP) samples from sickle cell disease subjects with known β-globin^{AT87Q} expression levels. Stimulated, untransduced SCD CD34⁺ cells, and DP lots listed in Table 3 were tested. Four samples (Group A) were chosen with VCN below, near, and above the low VCN limit of 0.8 c/dg. Six samples (Group C) were chosen with higher VCNs. Subject-matched, stimulated, untransduced samples, and DP samples were thawed and erythroid differentiated for 7 days in normoxia and 12 days in 2% O₂ for the Sickle Index method.

TABLE 3
Drug products

| | | | CoA VCN |
|---|---|---|---|
| Lot | Sample | Group | (c/dg) |
| 1 | 1 | A: bone marrow | 0.5 |
| 4 | 2 | derived | 1.3 |
| 2 | 3 | | 0.8 |
| 3 | 4 | | 0.9 |
| 6 | 5 | C: apheresis | 3.2 |
| 5 | 6 | derived | 2.8 |
| 9 | 7 | | 4.3 |
| 8 | 8 | | 4.0 |
| 10 | 9 | | 4.6 |
| 7 | 10 | | 3.3 |

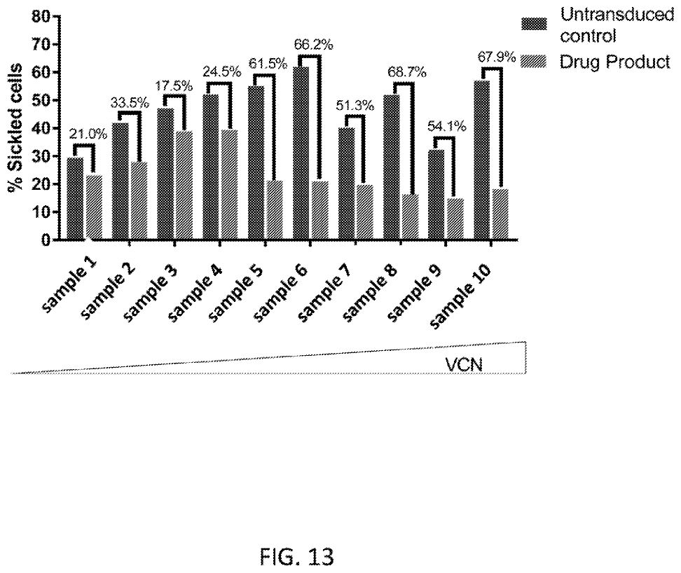

Potency using the Sickle Index method was between 51.3% and 68.7% for all six Group C samples (VCN 2.8 to 4.6 c/dg) (FIG. 13). In all six of the Group C samples, the abundance of sickled cells was reduced to 18.5%±2.6%, indicating that the assay is approaching the maximum observable anti-sickling effect of LentiGlobin BB305 in this method (Table 4). The abundance of sickled cells in the untransduced control varied considerably, substantiating the requirement for subject- and batch-matched untransduced comparator cells. For the Group A samples (VCN of 0.5 to 1.3 c/dg), the potency was substantially lower (17.5% to 33.5%), although still greater than the maximum non-specific potency of 15.8% observed previously (FIG. 12). However, the absolute potency of one drug product lot, Lot 1 (VCN=0.5 c/dg), was less than the minimum resolvable difference of 7.2% sickled cells described previously.

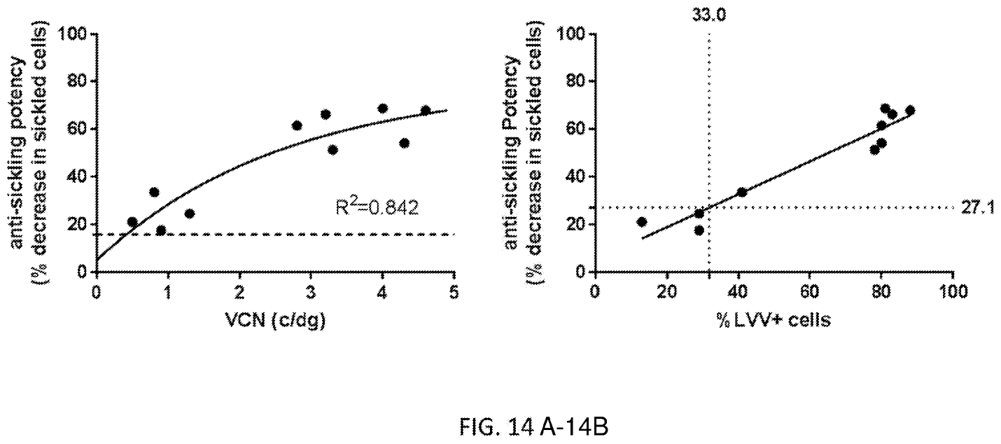

The relationship between VCN and potency by the Sickle Index method was fitted with an asymptotic equation (R2=0.842), consistent with the described maximization of potency at high VCNs (FIG. 14A). The asymptotic equation estimated the maximum anti-sickling potency observable with the Sickle Index method to be 79.6% reduction in sickled cells. The asymptotic fit is also consistent with the observed amount of sickled cells in the transduced samples (FIG. 13). Considering background non-specific potency (FIG. 12), the expected assay range is 15.8% to 79.6% reduction in sickled cells. The exponential fit further indicates that the assay is particularly sensitive at detection of potency in lots with VCNs less than 3.0 c/dg, whereas when VCNs greater than 3.0 c/dg the potency is maximized and indistinguishable among lots.

TABLE 4
Drug product potency using the Sickle Index method

| | | | % | | |
|---|---|---|---|---|---|
| | CoA | % | Sickled of | Absolute | % Potency |
| | VCN | Sickled of | LentiGlobin | % | relative to |
| Lot | (c/dg) | Untransduced | BB305 DP | Potency | UT control |
| 1 | 0.5 | 29.2% | 23.1% | 6.1% | 21.0% |
| 2 | 0.8 | 41.9% | 27.8% | 14.1% | 33.5% |
| 3 | 0.9 | 47.0% | 38.8% | 8.2% | 17.5% |
| 4 | 1.3 | 52.0% | 39.2% | 12.8% | 24.5% |
| 5 | 2.8 | 55.0% | 21.1% | 33.9% | 61.5% |
| 6 | 3.2 | 61.9% | 20.9% | 41.0% | 66.2% |
| 7 | 3.3 | 40.1% | 19.5% | 20.6% | 51.3% |
| 8 | 4.0 | 51.9% | 16.2% | 35.7% | 68.7% |
| 9 | 4.3 | 32.1% | 14.7% | 17.4% | 51.1% |
| 10 | 4.6 | 56.9% | 18.2% | 38.7% | 67.9% |

Unlike with VCN, comparing anti-sickling potency to % LVV+ cells reveals a linear relationship (FIG. 14B). Using the conversion from D14 VCN to % LVV+ cells, a VCN of 0.8 c/dg equated to 33.0% LVV⁺ cells. Therefore, based on our VCN specifications the potency specification would be expected to be roughly 27.1%.

Precision

Repeatability and intermediate precision were evaluated using the Sickle Index method with the expected % CV at or below 25%.

Precision of Sickle Index Analysis

Non-stimulated, untransduced SCD CD34⁺ cells from 3 lots were erythroid differentiated and analyzed by the Sickle Index using triplicate measurements by 2 operators. Each operator independently fixed and stained the erythroid differentiated cells from the three SCD cell lots in batches at two different timepoints. For instrument to instrument comparison, the same fixed and stained samples were run on both the Amnis ImageStream at site 1 and the Amnis ImageStream at site 2 one week apart using the same pre-designed acquisition template. Both samples were also analyzed using the same analysis template.

In all tested samples, the percentage of sickled cells measured ranged from 30.7 to 51.9% sickled cells, and the resulting CV of triplicates (0.8 to 8.8%) were consistent with the 3 to 6% CV observed in the dilutional linearity study (Table 2). Inter-operator precision was calculated for each sample, analyzed on the same instrument, with the second timepoint added such that three average values can be used to calculate % CV. Overall intermediate precision was calculated for each sample, across both operators, timepoints, and instruments. The results detailed in Table 5 and shown in Table 6, indicate that the overall intermediate precision of the Sickle Index is 5.8%, well below the expected maximum 25% CV.

TABLE 5
Precision of Sickle Index analysis

| Operator | 1 | | | | | | | |
|---|---|---|---|---|---|---|---|---|
| Timepoint | 1 | | | | 2 | | | |
| Instrument | 1 | | | | 1 | | | |
| Sample | Replicate | Value | Avg. | % CV | Replicate | Value | Avg. | % CV |
| 8 | 1 | 33.0 | 33.5 | 1.3% | 1 | 36.5 | 38.0 | 3.4% |
| | 2 | 33.5 | | | 2 | 38.3 | | |
| | 3 | 33.9 | | | 3 | 39.1 | | |
| 2 | 1 | 48.1 | 47.5 | 2.1% | 1 | 49.6 | 50.1 | 1.6% |
| | 2 | 48.0 | | | 2 | 49.7 | | |
| | 3 | 46.4 | | | 3 | 51.0 | | |
| 7 | 1 | 30.7 | 31.7 | 2.8% | 1 | 36.6 | 36.1 | 1.5% |
| | 2 | 32.2 | | | 2 | 35.5 | | |
| | 3 | 32.3 | | | 3 | 36.1 | | |
| Operator | 1 | | | | 2 | | | |
| Instrument | 2 | | | | 2 | | | |
| Sample | Replicate | Value | Avg. | % CV | Replicate | Value | Avg. | % CV |
| 8 | 1 | 33.3 | 33.6 | 0.8% | 1 | 35.8 | 36.4 | 1.5% |
| | 2 | 33.8 | | | 2 | 36.6 | | |
| | 3 | 33.7 | | | 3 | 36.8 | | |
| 2 | 1 | 47.1 | 47.5 | 1.0% | 1 | 49.2 | 49.3 | 0.6% |
| | 2 | 47.4 | | | 2 | 49.7 | | |
| | 3 | 48.0 | | | 3 | 49.1 | | |
| 7 | 1 | 35.6 | 33.7 | 5.0% | 1 | 38.3 | 36.6 | 5.1% |
| | 2 | 32.8 | | | 2 | 36.8 | | |
| | 3 | 32.6 | | | 3 | 34.6 | | |

| Operator | 2 | | | |
|---|---|---|---|---|
| Timepoint | 1 | | | |
| Instrument | 1 | | | |
| Sample | Replicate | Value | Avg. | % CV |
| 8 | 1 | 42.0 | 40.7 | 8.8% |
| | 2 | 43.5 | | |
| | 3 | 36.6 | | |
| 2 | 1 | 51.9 | 50.6 | 2.4% |
| | 2 | 50.6 | | |
| | 3 | 49.5 | | |
| 7 | 1 | 34.7 | 34.6 | 2.2% |
| | 2 | 35.2 | | |
| | 3 | 33.7 | | |
| Operator | 2 | | | |
| Instrument | 2 | | | |
| Sample | Replicate | Value | Avg. | % CV |
| 8 | 1 | 40.3 | 36.5 | 8.8% |
| | 2 | 35.0 | | |
| | 3 | 34.4 | | |
| 2 | 1 | 49.7 | 49.1 | 1.8% |
| | 2 | 48.1 | | |
| | 3 | 49.5 | | |
| 7 | 1 | 32.9 | 32.6 | 3.8% |
| | 2 | 33.7 | | |
| | 3 | 31.3 | | |

TABLE 6
Intermediate precision of Sickle Index analysis

| | | | | | Average |
|---|---|---|---|---|---|
| | | 1318 | 1320 | 1322 | CV |
| Amnis #1 | Timepoint CV | 7.3% | 3.4% | 7.3% | 6.0% |
| | Inter-operator | 9.9% | 3.4% | 5.9% | 6.4% |
| | CV | | | | |
| Amnis #2 | Timepoint CV | 4.5% | 2.2% | 6.4% | 4.4% |
| | Inter-operator | 6.1% | 2.1% | 6.6% | 4.9% |
| | CV | | | | |

| | | | | |
|---|---|---|---|---|
| Intermediate Precision | 8.5% | 2.9% | 6.1% | 5.8% |

Precision of Cell Culture and Sickle Index Analysis

The Sickle Index potency method uses a 19 day erythroid differentiation that could introduce significant biological variability to the assay. Therefore, the precision of the entire method, including the cell culture, needed to be evaluated. Because potency is calculated as a relative difference between transduced and untransduced cells, it was important to measure the CV among cell culture replicates and among resulting potency readouts, as factors such as media and cell culture could have similar effects on both transduced and untransduced cells that are cultured within each assay run.

To test the precision of cell culture, SCD CD34⁺ cells were transduced at MOI 25 (Pooled colony VCN=4.3 c/dg) and cryopreserved in 30 vials of 1×10⁶ cells in each. Untransduced comparator SCD CD34⁺ cells were stimulated for 48 hours and similarly cryopreserved. For each assay run, each operator thawed one vial of LentiGlobin BB305 transduced cells and one vial of stimulated, untransduced CD34⁺ cells. Fresh cell culture media was made for each assay run and each assay run started on a different week, capturing inter-day precision. The Sickle Index was acquired in triplicate on the Amnis ImageStream and analyzed by each operator using Amnis IDEAS. Assay repeatability, performed by operator 1 during run 1 using six replicates, indicated 4.8% CV in potency. The overall intermediate precision was also 4.8% CV, well below the expected 25% CV (Table 7, Table 8).

TABLE 7
Precision of cell culture and Sickle Index analysis

| | | | Control | | Transduced | | | |
|---|---|---|---|---|---|---|---|---|
| Operator | Run | Replicate | value | % CV | value | % CV | Potency | % CV |
| 1 | 1 | 1 | 51.8 | 2.6% | 17.3 | 9.0% | 66.6% | 4.8% |
| | | 2 | 52.6 | | 19.2 | | 63.5% | |
| | | 3 | 50.0 | | 19.3 | | 61.5% | |
| | | 4 | 51.3 | | 17.9 | | 65.0% | |
| | | 5 | 52.4 | | 15.4 | | 70.6% | |
| | | 6 | 49.3 | | 16.2 | | 67.2% | |
| | 2 | 1 | 48.6 | 5.1% | 18.3 | 3.4% | 62.4% | 2.8% |
| | | 2 | 53.0 | | 18.1 | | 65.9% | |
| | | 3 | 53.3 | | 19.3 | | 63.8% | |
| | 3 | 1 | 49.6 | 0.7% | 18.0 | 1.8% | 63.8% | 1.3% |
| | | 2 | 49.6 | | 18.6 | | 62.6% | |
| | | 3 | 49.0 | | 18.5 | | 62.2% | |
| 2 | 1 | 1 | 51.6 | 7.0% | 18.2 | 6.1% | 64.7% | 1.9% |
| | | 2 | 52.2 | | 19.4 | | 62.8% | |
| | | 3 | 45.9 | | 17.2 | | 62.5% | |
| | 2 | 1 | 49.7 | 1.3% | 14.3 | 6% | 71.3% | 2.9% |
| | | 2 | 49.3 | | 16.1 | | 67.3% | |
| | | 3 | 48.5 | | 14.8 | | 69.6% | |
| | 3 | 1 | 53.2 | 3.5% | 17.0 | 5.8% | 68.1% | 1.6% |
| | | 2 | 50.5 | | 15.1 | | 70.1% | |
| | | 3 | 54.1 | | 16.2 | | 70.0% | |

TABLE 8
Intermediate precision of cell culture and Sickle Index analysis
Untransduced CV
4.0%
Transduced CV
9.2%
Overall Potency CV
4.8%

System Suitability Controls

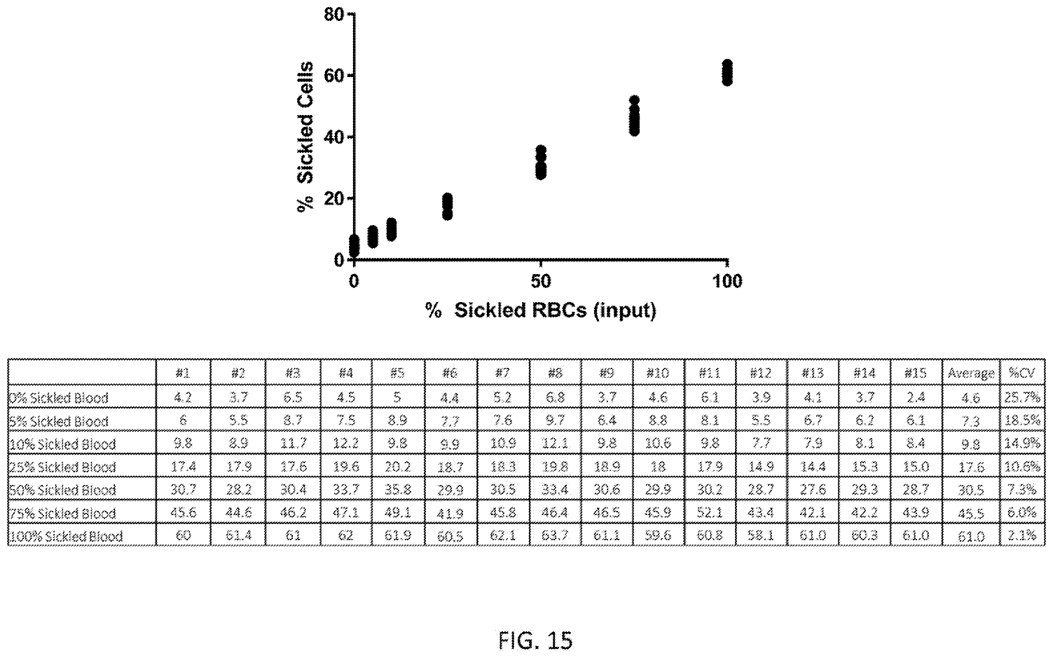

Aliquots of mixtures of healthy and sickled RBCs used for linearity study (Table 1) were run with each analysis of drug product retains or precision samples to verify that the Amnis ImageStream performance is suitable (FIG. 15). Across 15 experiments the highest CV was observed in samples with the smallest abundance of sickled cells. The proof of concept results (see above) indicated that the untransduced samples range from 50-60% sickled cells, whereas transduced samples contain approximately 20% sickled cells (Table 4), and therefore mixtures containing 25% and 100% sickled cells samples could be appropriate “low” and “high” system suitability controls.

The system suitability controls will be prepared using healthy donor blood and sickle donor blood that has been incubated at 2% O₂ for 20 hours ±2 hours and then fixed with 0.15% glutaraldehyde. The fixed sickle blood and healthy blood will then be counted and mixed at 25% sickled blood and 100% sickled blood and stored at 4° C.

What is claimed is:

1. A potency assay for a gene therapy treatment for sickle cell disease (SCD) comprising:
a) transducing a population of hematopoietic stem or progenitor cells from a subject that has sickle cell disease with a lentiviral vector comprising a polynucleotide encoding a globin;
b) performing two-phase erythroid differentiation of the population of hematopoietic stem or progenitor cells comprising culturing the hematopoietic stem or progenitor cells under hypoxia during erythroid differentiation;
c) fixing and staining the differentiated erythroid cells;
d) analyzing the fixed and stained erythroid cells with an imaging device;
e) calculating a Sickle Index value for the analyzed erythroid cells; and
f) calculating the percent of sickled erythroid cells in the population,
wherein the potency of the gene therapy treatment is the proportion of sickled cells in the population relative to an untransduced control.

2. The potency assay of claim 1, further comprising obtaining the hematopoietic stem or progenitor cells from the subject that has sickle cell disease.

3. The potency assay of claim 1 or claim 2, wherein the hematopoietic stem or progenitor cells comprise CD34⁺ cells.

4. The potency assay of any one of claims 1 to 3, wherein the hematopoietic stem or progenitor cells comprise CD133⁺ cells.

5. The potency assay of any one of claims 1 to 4, wherein the hematopoietic stem or progenitor cells comprise CD34⁺CD38^{Lo}CD90⁺CD45RA⁻ cells.

6. The potency assay of any one of claims 1 to 5, wherein the hematopoietic stem or progenitor cells comprise a pair of β-globin alleles selected from the group consisting of: β^{E}/β^{S}, β⁰/β^{S}, β^{C}/β^{S}, β⁺/β^{S} and β^{S}/β^{S}.

7. The potency assay of any one of claims 1 to 6, wherein the globin is a human β-globin, a human δ-globin, an anti-sickling globin, a human γ-globin, a human β^{A-T 87Q}-globin, a human β^{A-G16D/E22A/T87Q}-globin, or a human β^{A-T87Q/K95E/K120E}-globin protein.

8. The potency assay of any one of claims 1 to 7, wherein the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, or a derivative thereof.

9. The potency assay of any one of claims 1 to 8, wherein erythroid differentiation occurs in HiF erythroid differentiation media.

10. The potency assay of any one of claims 1 to 9, wherein erythroid differentiation occurs for a period of 21 to 25 days.

11. The potency assay of any one of claims 1 to 10, wherein erythroid differentiation occurs for a period of 21 days.

12. The potency assay of any one of claims 1 to 11, wherein the culturing of cells in the first phase of erythroid differentiation occurs under normoxia conditions.

13. The potency assay of any one of claims 1 to 12, wherein the culturing of cells in the first phase of erythroid differentiation occurs under normoxia for a period of 1-6 days.

14. The potency assay of any one of claims 1 to 13, wherein the culturing of cells in the second phase of erythroid differentiation occurs under hypoxia conditions.

15. The potency assay of claim 14, wherein the hypoxia conditions comprise 2% O₂.

16. The potency assay of claim 14 or claim 15, wherein the hypoxia conditions comprise 2% O₂ and 5% CO₂.

17. The potency assay of any one of claims 1 to 16, wherein the culturing of cells in the second phase of erythroid differentiation occurs under hypoxia conditions for a period of 1-15 days.

18. The potency assay of any one of claims 1 to 16, wherein the culturing of cells in the second phase of erythroid differentiation occurs under hypoxia conditions for a period of 1-12 days.

19. The potency assay of any one of claims 1 to 16, wherein the culturing of cells in the second phase of erythroid differentiation occurs under hypoxia conditions for a period of 12 days.

20. The potency assay of any one of claims 1 to 19, wherein an erythroid differentiation medium is switched to Iscove's Modified Dulbecco's Medium (IMDM) on day 12 of the second phase of erythroid differentiation, and wherein the cells are incubated under hypoxia conditions for at least 15 hours.

21. The potency assay of any one of claims 1 to 20, wherein the cells are fixed under hypoxia conditions.

22. The potency assay of any one of claims 1 to 21, wherein the differentiated erythroid cells are stained with thiazole orange.

23. The potency assay of any one of claims 1 to 22, wherein the imaging device is a flow cytometry device.

24. The potency assay of claim 23, wherein the flow cytometry device is an Amnis ImageStream device.

25. The potency assay of any one of claims 1 to 24, further comprising calculating the shape ratio for the fixed and stained erythroid cells, wherein the shape ratio is calculated as the minimum thickness of the cell divided by the length of the cell.

26. The potency assay of any one of claims 1 to 25, wherein the Sickle Index value is calculated as the shape ratio divided by the area of each cell, and wherein the shape ratio is calculated as the minimum thickness of the cell divided by the length of the cell.

27. The potency assay of any one of claims 1 to 26, wherein the percent of sickled erythroid cells is calculated by identifying the percent of erythroid cells in the population having a Sickle Index value less than 0.004.

28. The potency assay of any one of claims 1 to 27, further comprising analyzing untransduced cells in a second population of hematopoietic stem or progenitor cells from the subject with the imaging device.

29. The potency assay of claim 28, further comprising calculating the shape ratio for the untransduced cells, wherein the shape ratio is calculated as the minimum thickness of the cell divided by the length of the cell.

30. The potency assay of claim 28 or claim 29, further comprising calculating a Sickle Index value for the untransduced cells, wherein the Sickle Index value is calculated as the shape ratio divided by the area of each cell, and wherein the shape ratio is calculated as the minimum thickness of the cell divided by the length of the cell.

31. The potency assay of any one of claims 28 to 30, further comprising calculating the percent of sickled untransduced cells, wherein the percent of sickled untransduced cells is calculated by identifying the percent of untransduced cells in the second cell sample having a Sickle Index value less than 0.004.

32. The potency assay of claim 31, further comprising calculating the relative potency of gene therapy treatment, wherein the relative potency is calculated as the percent sickled untransduced cells minus the percent sickled transduced cells divided by the percent sickled untransduced cells.

33. A method for measuring relative potency of a drug product comprising:
a) calculating a Sickle Index value for a first population of hematopoietic stem or progenitor cells transduced with a lentiviral vector comprising a polynucleotide encoding a globin and for a second population of untransduced hematopoietic stem or progenitor cells, wherein the formula for calculating the Sickle Index value is:
$$Sickle Index = \frac{( minimum thickness length of each cell )}{area of each cell} ;$$
b) identifying the percent of sickled cells in a sample, wherein the cells are considered to be sickled if the Sickle Index value is less than 0.004; and
c) calculating the relative potency of the drug product, wherein the formula for calculating relative potency is:
$$Relative Potency \% = \frac{( \% sickled untransduced - \% sickled transduced )}{\% sickled untransduced} ,$$
wherein the first population and the second population are obtained from a patient having sickle cell disease.

34. The method of claim 33, further comprising obtaining the cells from the patient having sickle cell disease.

35. The method of claim 33 or claim 34, wherein the hematopoietic stem or progenitor cells comprise CD34⁺ cells.

36. The method of any one of claims 33 to 35, wherein the hematopoietic stem or progenitor cells comprise CD133⁺ cells.

37. The method of any one of claims 33 to 36, wherein the hematopoietic stem or progenitor cells comprise CD34⁺CD38^{Lo}CD90⁺CD45RA⁻ cells.

38. The method of any one of claims 33 to 37, wherein the hematopoietic stem or progenitor cells comprise a pair of β-globin alleles selected from the group consisting of: β^{E}/β^{S}, β⁰/β^{S}, β^{C}/β^{S}, β⁺/β^{S} and β^{S}/β^{S}.

39. The method of any one of claims 33 to 38, wherein the globin is a human β-globin, a human δ-globin, an anti-sickling globin, a human γ-globin, a human β^{A-T87Q} globin, a human β^{A-G16D/E22A/T87Q}-globin, or a human β^{A-T87Q/K95E/K120E}-globin protein.

40. The method of any one of claims 33 to 39, wherein the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, or a derivative thereof.

41. The method of any one of claims 33 to 40, wherein the Sickle Index value is calculated using a flow cytometry device.

42. The method of claim 41, wherein the flow cytometry device is an Amnis ImageStream device.

43. The method of any one of claims 33 to 42, wherein the population of hematopoietic stem or progenitor cells transduced with the lentiviral vector are differentiated using a two-phase erythroid differentiation protocol before the Sickle Index value is calculated.

44. The method of claim 43, wherein the second phase of the erythroid differentiation protocol occurs under hypoxia conditions.

45. The method of claim 44, wherein the hypoxia conditions comprise 2% O₂.

46. The method of claim 44 or claim 45, wherein the second phase of the erythroid differentiation protocol occurs for a period of 1 to 15 days.

47. The method of claim 44 or claim 45, wherein the second phase of the erythroid differentiation protocol occurs for a period of 12 days.

48. The method of claim 43, wherein the erythroid-differentiated hematopoietic stem or progenitor cells are fixed under hypoxia and stained with thiazole orange.
